(19) United States
(12) Patent Application Publication
(10) Pub. No.: US 2026/0207859 A1
(43) Pub. Date: Jul. 23, 2026

(54) HYPODERMIC NEEDLES AND METHODS OF MANUFACTURE

(71) Applicant: VenoCare, Inc., Doral, FL (US)

(72) Inventors: Raul LEYTE-VIDAL, Doral, FL (US); Amir BELSON, Savyon (IL)

(21) Appl. No.: 19/139,315

(22) PCT Filed: Dec. 15, 2023
(86) PCT No.: PCT/US2023/084427

§ 371 Date: Jun. 13, 2025

Related U.S. Application Data
(60) Provisional application No. 63/387,688, filed on Dec. 15, 2022.

(51) Int. Cl. A61M 5/32

(52) U.S. Cl. CPC A61M 5/3286

(57) ABSTRACT

Hypodermic needles are described with a proximal end, a distal end with a tissue penetrating tip and a lumen extending from the proximal end to the distal end. The elongate body may have a channel formed in an exterior surface. The channel may be concave or formed in an outer wall. The portion having the channel may maintain a circular lumen or the lumen may be non-circular or confirming to a shape of the channel. Additionally, the elongate body may a flat section along a length of the elongate body. The flat section may be at the distal most portion of the elongate body extending proximally from the distal tip. The elongate body may also include a notch formed in an outer wall to provide communication with or is open to the lumen. The notch opening may be transverse to the lumen or be aligned with the lumen.

### CROSS REFERENCE TO RELATED APPLICATIONS

This patent application claims priority to U.S. Provisional patent application Ser. No. 63/387,688, filed Dec. 15, 2022, titled “HYPODERMIC NEEDLES AND METHODS OF MANUFACTURE,” which is incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present invention relates generally to hypodermic needles. More particularly, the present invention provides a hypodermic needle with one or more flat surfaces along a length of the needle to improve vascular access and intravascular medical procedures.

### BACKGROUND

Vascular access is necessary element of medical procedures from catheter placement and blood draws to delivering fluids and drugs. Aside from invasive surgical procedures to access vasculature, needles were developed in the early part of the 19th century to provide vascular access with the least amount of physical damage to a patient. Since that time, there has been little innovation outside of different alloys or compositions for these needles. There has been a stark difference in the amount of innovation between the medical devices and tools coupled to, or relying on, needles compared to the needles themselves.

An example of a common medical procedure requiring hypodermic needles is venipuncture for vascular access. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like. While venipuncture in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

The difficult access emphasizes the need for an improved needle to increase opportunity for additional capabilities of associated medical devices and tools. Once the vessel has been accessed, catheter placement may require use of a guide wire or other type of guide tool to advance the catheter. Current needles require complex and cumbersome procedures of inserting, removing, re-inserting, adjusting, advancing, retracting, of various catheters, guide tools, etc. In addition to all of the tools and devices, the procedures for their use can be complex and introduce opportunity for error or increased injury to the patient.

For these reasons, it would be desirable to provide improved devices, methods and systems for improving (e.g., simplifying) vascular access and reducing the negative impact on the patient. It would be particularly desirable to provide an improved hypodermic needle that can be configured to improve and expand the function and operation of associated tools and devices during medical procedures. At least some of these objectives will be met by the various embodiments that follow.

### SUMMARY OF THE DISCLOSURE

In one aspect, there is provided a hypodermic needle with an elongate body having a proximal end and a distal end; a tissue penetrating tip at the distal end; a lumen extending through the elongate body from the proximal end to the distal end; and a channel formed along a length of the elongate body. In one embodiment, the channel is formed by a press fit depression in a surface of the elongate body. In another aspect, the lumen adjacent to the channel has a complementary shape to the depression in a surface of the elongate body. In over versions, there is a transition segment positioned between a proximal length of the elongate body and the channel. In still other aspects, there is a transition segment positioned at a proximal end of the elongate body, wherein the channel extending proximally from the transition segment is concave relative to the length of elongate body proximal to the transition segment. Another variation includes a proximal transition segment, a distal transition segment, wherein the channel further comprises a proximal channel and a distal channel further wherein the distal transition segment is positioned between the proximal channel and the distal channel. In still other aspects, the elongate body further comprises a proximal region and a distal region wherein the proximal region and the distal region are separated by a transition segment. Optionally, the transition segment comprises a surface forming an acute angle with a surface of the channel. In still other alternatives, the channel extends along an entire length of the elongate body. There are other configuration having a transition segment separates the proximal channel from the distal channel, wherein the distal channel is more concave than the proximal channel. In one embodiment, the lumen is non-circular, in another the lumen conforms to a shape of an adjacent portion of a channel. In another aspect, there is a notch extending through the elongate body into a portion of the lumen. In another variation, the tissue penetrating tip is positioned on the distal end of the elongate body opposite the channel. Additionally or optionally, the channel includes a distal channel and a proximal channel, wherein the distal channel extends between the elongate body distal end and a transition segment, and the proximal channel extends from the transition segment to the elongate body proximal end wherein the depth of the distal channel is greater than the depth of the proximal channel. There is another variation where the channel is crescent shaped and formed in an outer wall of the elongate body and the lumen has a corresponding crescent shape to the channel crescent shape.

In another embodiment, there is a hypodermic needle having an elongate body having a proximal end and a distal end; a tissue penetrating tip positioned at the distal end; a lumen extending from the proximal end to the distal end; a proximal channel extending along a proximal portion of the elongate body; and a distal channel extending from a distal portion of the elongate body to a transition segment that separates the proximal channel from the distal channel. In one variation, relative to an outer wall of the elongate body the distal channel to is deeper than the proximal channel. In still another alternative, there is a proximal transition segment at the proximal end of the proximal channel separating the proximal channel from an outer wall of the elongate body. In another configuration, the proximal channel and the distal channel are pressed into the elongate body. Still another variation has a proximal transition segment, a distal transition segment and an annular shaped proximal portion of the elongate body adjacent to the proximal transition segment. In other aspects, the proximal channel extends distally from the proximal transition segment. In another configuration, the distal channel extends distally from the distal transition segment. In yet another configuration, the proximal channel extends distally from the proximal end of the elongate body. Optionally, the proximal channel extends from a transition segment positioned distal to the elongate body proximal end.

In another embodiment, there is a hypodermic needle having an elongate body having a proximal end and a distal end; a tissue penetrating tip at the distal end; a lumen extending through the elongate body from the proximal end to the distal end; a channel formed along a length of the elongate body; and a notch formed in a wall of the elongate body, the notch forming an opening into the lumen. In one alternative, the wall of the elongate body has a curved external shape or an annular external shape. In another aspect, the wall of the elongate body has a flat shape and the remainer of the elongate body has a curved external shape or an annular external shape. In one variation the notch is transverse to a longitudinal axis of the lumen. In another configuration, the notch is aligned with a longitudinal axis of the lumen. In various alternatives the shape of the opening formed by the notch is rectangular, curved, v-shaped, or faceted.

In yet another alternative embodiment, there is a hypodermic needle with an elongate body having a proximal end and a distal end; a tissue penetrating tip at the distal end; a flat section at the distal end; a lumen extending through the elongate body from the proximal end to the distal end; a channel formed in an outer wall of the elongate body, the channel formed along a length of the elongate body proximal to the flat section; and at least one notch forming an opening into the lumen. In one variation, the notch is transverse to a longitudinal axis of the lumen. In another optional configuration the notch is aligned with a longitudinal axis of the lumen. In other configurations the shape of the opening formed by the notch is rectangular, curved, v-shaped, or faceted. Additionally or optionally, the at least one notch comprises a notch on the flat section and a notch on the channel. In still another configuration, the elongate body is annular, and the channel forms a concave surface in the elongate body. In still another variation, the lumen has a circular cross section proximal to the flat section.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is a perspective view of an embodiment of a hypodermic needle from a distal end, as described herein.

FIG. 2A is a side elevation view of the hypodermic needle shown in FIG. 1.

FIGS. 2B, 2C, and 2D are additional views of the hypodermic needle shown in FIG. 2A showing details of the distal section, distal transition section, and proximal transition section, respectively.

FIG. 3A is a perspective view of the hypodermic needle shown in FIG. 1 from the distal end.

FIG. 3B is another perspective view of a distal segment of the hypodermic needle from FIG. 3A shows examples of a notch in a bottom surface of the hypodermic needle.

FIG. 4 is a side elevation view of the hypodermic need from FIG. 1.

FIG. 5 is profile view from the distal end of the hypodermic needle shown in FIG. 1.

FIG. 6A is a side elevation view of an embodiment of hypodermic needle, as described herein.

FIG. 6B is a detailed view of a proximal portion of the hypodermic needle shown in FIG. 6A.

FIG. 7A is a side elevation view of a hypodermic needle, as described herein.

FIG. 7B is a detailed view of a distal portion of the hypodermic needle shown in FIG. 7A.

FIGS. 8A and 8B are perspective views of distal end examples from hypodermic needles described herein.

FIGS. 9A to 9D are perspective views showing examples of hypodermic needles as described herein.

FIG. 10A is a perspective view of an example of a hypodermic needle as described herein.

FIG. 10B is a perspective view of an example of a hypodermic needle as described herein.

FIG. 11 is a perspective view of an example of a hypodermic needle distal end as described herein showing details of the needle distal end and tissue penetrating tip.

FIG. 12A is a perspective view of a hypodermic needle showing an exemplary configuration and arrangement of features as described herein.

FIGS. 12B and 12C are detailed perspective views of the hypodermic needle from FIG. 12A with the distal segment shown in FIG. 12B and the proximal segment shown in FIG. 12C.

FIGS. 13A to 13L show detailed views of distal section examples of hypodermic needles described herein with different notch configurations.

FIGS. 13A-13D are side views of a notch that is transverse to a lumen of the needle having a rectangular, curved, v-shaped or faceted shape, respectively, formed in a flat surface of the needle.

FIG. 13E is a top down view of a notch with a rectangular shape along the needle lumen.

FIG. 13F is a top-down view of a circular notch in a flat surface of the needle.

FIGS. 13G-13J are notches open to the lumen similar to FIGS. 13A-13D with the exception that the notch is formed in a curved or annular portion of the needle body or a surface opposite to the flat needle surface.

FIG. 13K is a bottom-up view of a notch with a rectangular shape along the needle lumen, where the notch is formed in a rounded wall of the needle. The notch may be on a side opposite to a flat surface of the needle.

FIG. 13L is a bottom-up view of a circular notch in a rounded wall of the needle. The notch may be formed in a rounded wall portion that is opposite to a flat wall of the needle.

### DETAILED DESCRIPTION

Needles described herein can have features for use in combination with guide elements or other tools adapted to extend along the needle exterior surface. Generally a needle may have an elongate body or cannula with a lumen extending therethrough from a proximal end to a distal end to facilitate a flow of fluid therethrough. The distal end of a needle can have a tissue penetrating tip adapted to penetrate tissue of a patient during use. The exterior surface of a needle may have one or more channels extending along a length of the needle body including along the entire length of the elongate body from the proximal end to the distal end. In some examples, the needle may have one or more channels extending along a length of the elongate body less than the entire length. For example, a needle described herein may have one or more channels extending along a segment of the elongate body.

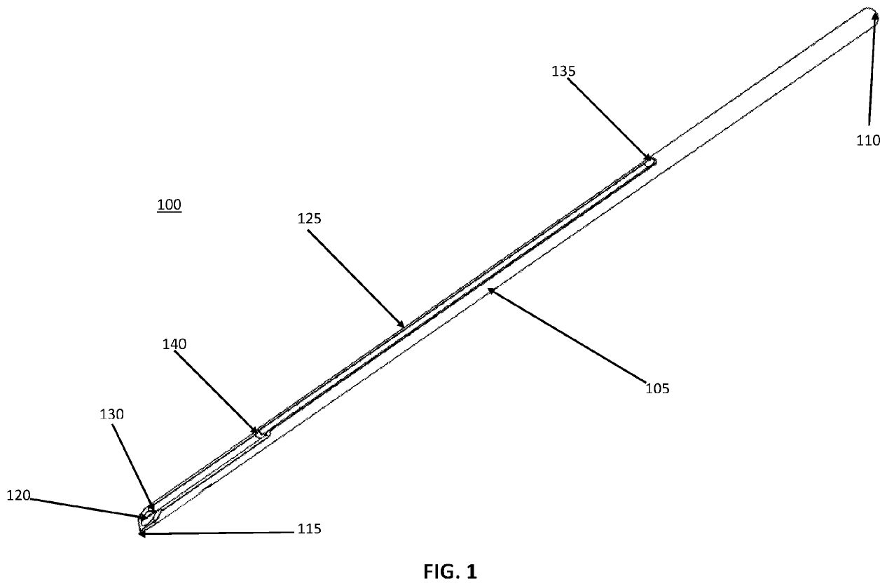

FIG. 1 shows an example of a needle 100 in a perspective view from the distal end. The elongate body 105 extends between the needle proximal end 110 and the tissue penetrating tip 115 at the distal most end of the needle. The proximal end of lumen 120 extending through the elongate body 105 is shown and can be adapted to direct a flow of fluid (e.g., blood) through the elongate body 105. In this example, a channel 125 is shown along an exterior surface of the needle 100. The channel 125 is generally concave and may be pressed into the needle during manufacture such that the elongate body is changed from a cylindrical cannula to having one or more channels along the exterior of the elongate body 105. The channel distal end 130 is shown in FIG. 1 on an opposite side of the tissue penetrating tip 115 and forms a segment of the distal perimeter of the lumen 120.

In some examples, the channel may be a concave channel with a uniform depth. Channels described herein may extend along a length of the needle (e.g., elongate body). For example, a channel may extend along the entire length of the elongate body from the proximal end (e.g., proximal lumen perimeter) to the distal end (e.g., distal lumen perimeter). In some examples, channels described herein may have a uniform depth along an entire length of the channel. In some examples, channels described herein may have a first depth at a proximal end of the channel and second depth at the distal end of the channel. For example, at or near the distal end of a channel, the depth of the channel may be greater than the depth at or near the channel proximal end. In some examples, the at or near the distal end of a channel, the depth of the channel may be less than the depth at or near the channel proximal end. In some examples, the channel may be described in terms of channel segments. In some examples, each channel segment may have the same depth. In some examples, one channel (e.g., channel segment) may have a greater depth than another channel (e.g., channel segment).

The example shown in FIG. 1 illustrates an example of a needle with channel segments separated by transition regions 135 and 140. In this example, the proximal transition segment 135 is positioned between the channel 125 and a proximal portion of the elongate body extending to the proximal end 110. As illustrated in this example, the elongate body proximal to the transition segment 135 is generally cylindrical and the transition segment 135 is positioned between the cylindrical exterior of the proximal end of the elongate body 105 and the channel 125 extending distally from transition segment 135.

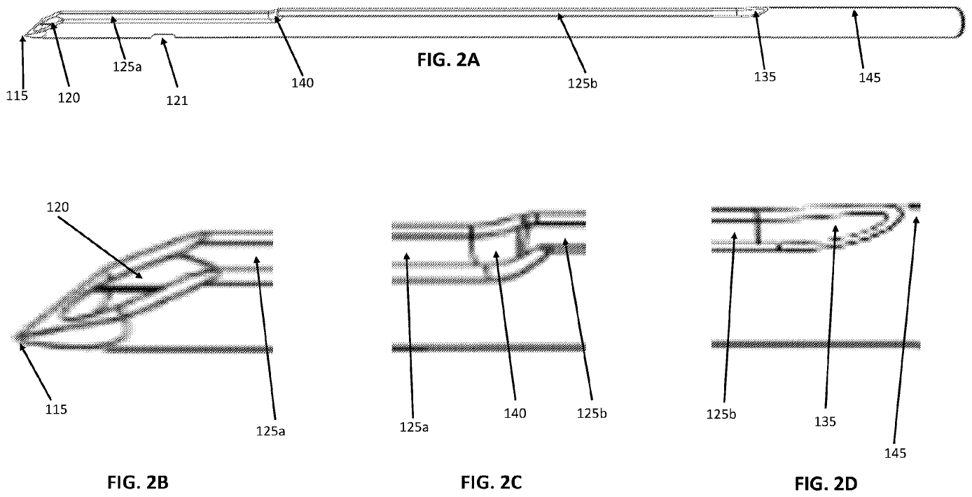

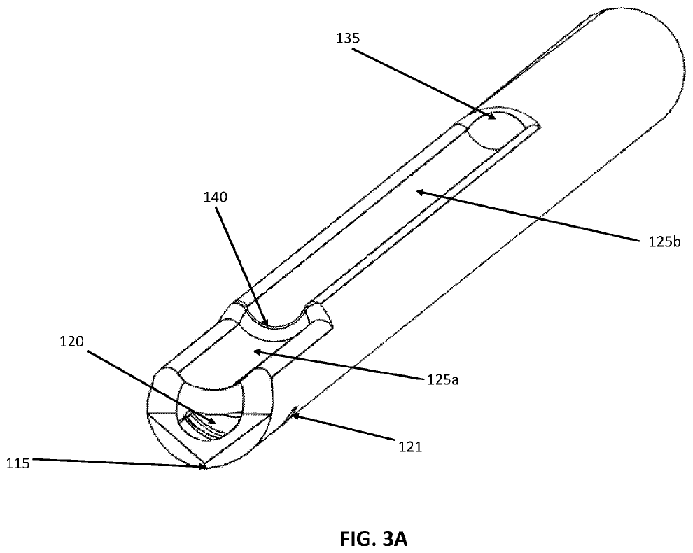

FIG. 2A shows the needle from FIG. 1 in a side elevation view having the proximal channel 125b and distal channel 125a along a segment of the overall needle length. In this example, the channel 125 may comprise two channel segments 125a and 125b. From the elongate body proximal end 110, the proximal transition segment 135 is initially pressed into the elongate body forming a transition from the needle proximal portion to the first (e.g., proximal) channel segment 125b. The notch 121 can also be seen in this view and is generally positioned proximal to the tissue penetrating tip 115 and on the opposite side of the needle from the channel. FIG. 2B shows a detailed view of the distal end of the needle from FIG. 2A to illustrate details of the distal channel 125a transition to the distal end of the needle and forming a portion of the lumen perimeter. In FIG. 2C, details of the transition segment 140 positioned between the distal channel 125a and the proximal channel 125b are illustrated.

In FIG. 2D, the proximal transition segment 135 is detailed whereby channel segment 125b is shown extending distally from transition segment 135 to the distal transition segment 140 positioned between channel segment 125b and 125a that extends distally from transition segment 140. As shown in this example, the distal channel segment 125a is deeper (e.g., pressed further into the elongate body) than channel segment 125b. Changes in the depth of a channel or channel segment may be adapted to accommodate different segments, features, regions, etc. of guide elements or other tools that may be extended along the needle exterior surface during use. For example, as illustrated in FIGS. 2A to 2D, the distal channel segment 125a being deeper than the proximal segment 125b can be adapted to accommodate a distal portion of a guide element that may be larger than the guide element elongate body extending proximally therefrom.

In some examples, transition segments described herein may be configured to provide a gradual transition of a channel or channel segments along a length of the elongate body. In some examples, transition segments may be adapted to accommodate a guide element or similar tool while motivating and maintain position of the guide element or tool along the needle exterior. For example, a transition segment with a deeper channel extending proximally therefrom may be adapted to prevent a segment of guide element positioned distal to the transition segment from retracting proximally against the transition segment. For example, deployment of a guide element in this example may allow for the guide element to be advanced along the channel to deploy a larger distal segment of the guide element from a distal channel segment.

In some examples, needles described herein may have a channel adapted to receive a guide element extending along the exterior surface of the needle. For example, a channel may be adapted to accommodate a guide element or guide element segment for routing or providing a path for the guide element.

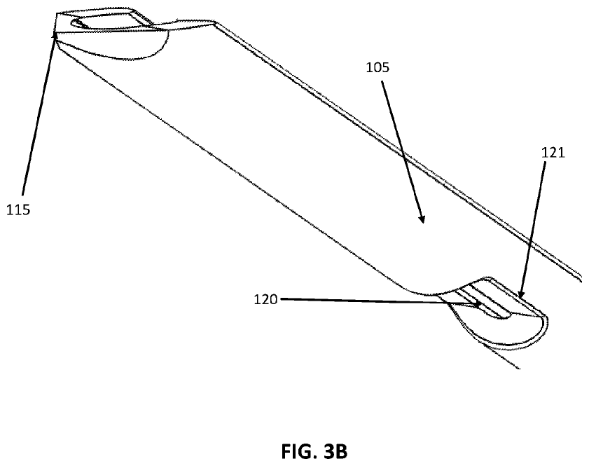

FIG. 3A shows another perspective view of a needle described herein from the distal end. Here, the distal end of the lumen 120 is shown and in this perspective a notch (e.g., bleed-back feature) 121 can be seen extending through a wall of the needle (e.g., elongate body) to expose the lumen to the exterior of the needle. In some examples, needles described herein may have one or more notches (e.g., notch 121) positioned on the needle and adapted to expose the needle lumen such that the flow of blood through the needle lumen can be confirmed when a portion of the blood is visible through the notch. For example, the notch may provide a visual indication of successful penetration of a blood vessel when blood is seen through the notch 121. FIG. 3B illustrates a perspective view of the needle from FIG. 3A from a bottom side of the distal portion of the needle. The notch 121 is positioned on the bottom (e.g., underside) of the needle and is configured to expose a portion of the lumen 120 to allow for visual confirmation of the presence of blood in the needle lumen 120.

The lumen 120 can be seen having a generally crescent shape cross section. Channel segment 125a extends along a distal length of the needle body and the lumen 120 is compressed from a cylindrical chape to the crescent shape shown in FIG. 3A. In some examples, needles described herein with one or more channels may be manufactured by compressing the concave channel into a cannula (e.g., needle elongate body) such that the lumen is adapted from a generally cylindrical lumen before the channel to a crescent shape corresponding to the depth of the channel pressed into the elongate body. For example, the crescent characteristics of a needle lumen may be based on the concave characteristics (e.g., channel depth, width, position, etc.) of the channel. In some examples, the thickness of the walls of a needle elongate body may be sufficient to allow for creation of a channel by grinding or removing material from the needle wall to form the channel. In this example, the lumen of a needle described herein may have one or more channels (e.g., channel segments) with a circular cross section uniform from the distal end of the elongate body to the proximal end of the elongate body.

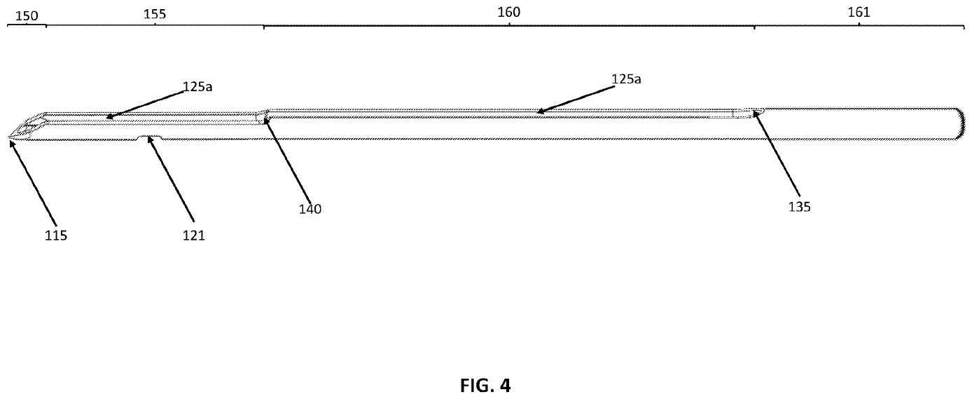

Referring to FIG. 4, the side elevation view of the needle 100 shows examples of needle regions or segments distinguishable by different channel segments, features, or characteristics of the elongate body. As described above, the distal channel segment 155 is deeper than the channel segment 166. In this example, the lumen extending from the distal end 150 to the proximal end may have a different diameter along segment 155 compared to segment 160. In some examples, both segments 155 and 160 may have a different diameter relative to the proximal position of a needle without a channel segment. A channel may be positioned along any length of the elongate body and may have a length corresponding to a guide element or other tool for which the needle is adapted to accommodate on an exterior surface. For example, the channel segment length 155 may be the same, greater than, or less than the channel segment length 160. The example illustrated in FIG. 4 may be configured to accommodate a guide element having a distal end positionable on the distal channel along length 155 and a guide element proximal end positionable along channel segment length 160. In some examples, the needles described herein may be configured to accommodate a guide element having a distal segment that is larger or includes features that benefit from a deeper channel in the needle exterior surface.

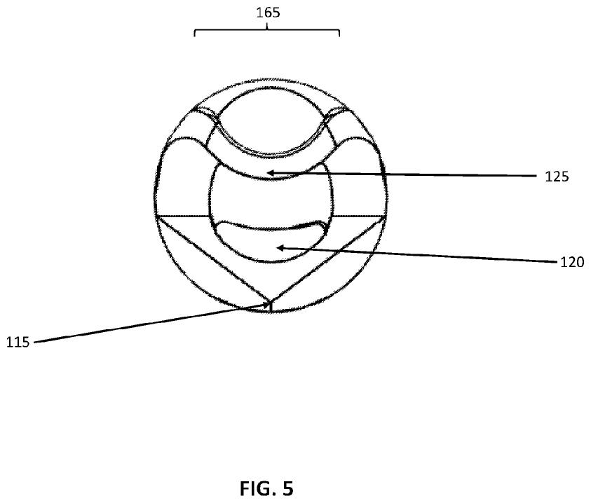

In FIG. 5, the needle 100 is shown from a profile view looking through the lumen 120 at the distal end. The tissue penetrating tip 115 is on the distal lumen perimeter generally opposite the channel 125. In this view, the width of a channel can be appreciated and understood as adaptable or configurable to accommodate a guide element or other tool therein. For example, where a guide element may have a distal end or segment that is larger than the guide element elongate body, the width of a distal channel segment may be larger than proximal channel segments to accommodate the larger portion of the guide element therein. Also in FIG. 5, an example of the crescent shape of lumen 120 is visible having a cross section corresponding to the depth of the channel 125.

In some examples, the lumen may extend linearly from the distal end of a needle through to the proximal end. In some examples, the lumen may curve or otherwise be non-linear along its entire length as the changes in channel depth may provide for a change in the lumen. For example, the lumen may be crescent shaped at or near the distal end of the needle and may be cylindrical at or near the proximal end of the needle with a transition from a crescent shaped lumen to a cylindrical lumen. In some examples, a lumen described herein may be coaxial along an entire length of the lumen through a needle. In some examples, a lumen may not be coaxial along the entire length of the needle where there are multiple channel segments and/or cylindrical needle segments. For example, referring back to FIG. 4, from the needle proximal end to the needle distal end, the lumen may have a first axis from the needle proximal end until the first channel (e.g., length 160) where the lumen axis may change to a second lumen axis along length 160, where the second lumen axis may change to a third lumen axis along length 150. In some examples, even though a needle may comprise a channel or multiple channel segments, the lumen may be coaxial from the needle distal end to the needle proximal end.

In some examples, a needle (e.g., hypodermic needle, access needle, transcutaneous needle, sharps, etc.) has a tissue penetrating end and an engagement element separated by an elongate body having at least one flat surface along a length of the elongate body. A central lumen may extend from the proximal end to the distal end and be configured to direct a flow of one or more fluids therethrough. The tissue penetrating distal end can be configured to penetrate one or more layers of tissue when advanced through contact with an exterior surface of a substrate (e.g., biological tissue). In some examples, the needles described herein may comprise one or more flat surfaces, one or more channels, or a combination of one or more flat surfaces and one or more channels positioned along a length of the elongate body. For example, a needle may comprise a channel (e.g., concave channel) along a length of the elongate body proximal to a flat surface extending distally beyond the concave channel. In some examples, a needle may comprise a concave channel extending distally along a length of the elongate body from a flat surface. In some examples, the arrangement of a flat surface and a concave channel may be in any arrangement along a length of the exterior of the elongate body.

In some examples, a hypodermic needle as described herein may have one or more flat surfaces that can extend along a length of the needle. In some examples, the length of the needle may be the distance of the cannula from a proximal end to the tissue penetrating tip. In some examples, the length of a needle may be the distance from the distal end to the proximal end, which may comprise one or more engagement elements. For example, the length of the needle may comprise a distance from the distal end to an engagement element configured to engage a cannon (e.g., cannula-cannon junction) Considering an example of the length of a hypodermic needle described herein being defined by a longitudinal distance between the proximal end and the distal end (e.g., tissue penetrating end), the flat surface may extend longitudinally along the length of the needle. In some examples, a flat surface may extend along the entire length of the needle. In some examples, a flat surface may extend along a length of the needle (e.g., cannula) less than the entire length of the needle.

The flat surface can be configured to facilitate the passage of one or more tools along a length of the flat surface during use. In some examples, a hypodermic needle described herein may be operably coupled to one or more medical devices (e.g., tools). In some examples, the coupled medical device may rely on the hypodermic needle for penetration and access to tissue, vasculature, cavities, other anatomy or a combination thereof. In some examples, the coupled medical device may rely on the hypodermic needle for penetration and access through tissue, vasculature, cavities, other anatomy or a combination thereof. The flat surface can be configured to reduce a cross-sectional surface area and thereby reduce the physical insult to tissue during use.

In some examples, the flat surface may be a bearing surface against which one or more tools may slidingly contact. A hypodermic needle as described herein may be configured to engage a medical device. For example, an engagement element locatable at a proximal end of the hypodermic needle may be coupled, affixed, adhered, integrated, etc. to a medical device. Some examples of a medical device may be a syringe, barrel, tube, hub, catheter, intravascular access device, etc. An engagement element at the proximal end of the hypodermic needle may be configured to engage and retain the needle in operable communication with the medical device. In some examples, the hypodermic needle may be configured to facilitate the transmission, flow, passage of one or more fluids from the medical device to the patient (e.g., the vessel or tissue where the hypodermic needle is positioned).

The flat surface along the needle can be configured to contact or otherwise engage one or more tools. For example, a hypodermic needle described herein may have one or more flat surfaces along a length of the needle and each flat surface may be configured to accommodate, contact, or otherwise engage a tool. In some examples, the needle may be used in combination with one or more components (e.g., medical devices) during a procedure involving tissue penetration. One example of a medical device may be an intravascular access device that may rely on the needle for initial access to a blood vessel and introduction of a catheter, introducer, dilator, etc. In such an example, the needle may be locatable within a catheter lumen at a distal end of the intravascular access device.

A tissue penetrating end may comprise a tip configured to penetrate tissue of a patient during use (e.g., a tissue penetrating tip) The tissue penetrating tip may me locatable at a distal end of a bevel surface extending from one or more flat surfaces along the needle. The tissue penetrating tip can be configured to penetrate one or more tissues. In some examples, the tissue penetrating tip can be an apex of one or more surfaces that may converge with one another at a point (e.g., tissue penetrating tip). In some examples, the tissue penetrating tip may comprise an apex of a plurality of flat surfaces (e.g., a polyhedron). In some examples, the tissue penetrating tip may be the apex of a pyramidal arrangement of three or more surfaces. In some examples, the surfaces converging to the apex (e.g., tissue penetrating tip) may be secondary, tertiary, quaternary, etc. bevel surfaces. A number of bevel surfaces on the tissue penetrating end may be configured to minimize the physical injury to the tissue as the tissue penetrating tip advances therethrough.

In some examples, a bevel surface of the needle distal tip may be flat or substantially flat. In some examples, a bevel surface of the needle distal end may comprise one or more curves, arcs, or other feature associated with a non-flat surface. For example, a bevel surface may be concave from the proximal end of the bevel surface to the distal end of the bevel surface (e.g., the tissue penetrating tip). In some examples, a bevel surface may be convex or otherwise curved outward from the proximal end of the bevel surface to the distal end of the bevel surface (e.g., the tissue penetrating tip.

In some examples, a tissue penetrating tip may comprise a single bevel, multi-bevel, multi-facet configuration. The tissue penetrating tip may have one or more geometric features of configurations distal of the flat surface along the cannula, as described herein. The geometry of the tissue penetrating tip may be based on the application for which the needle is being used. Some examples of a hypodermic needle tissue penetrating tip, as described herein may include one or more elements or geometric features from needle tip configurations such as a diamond needle tip, franseen needle tip of a quincke needle tip, whitacre needle tip, sprotte needle tip, short bevel needle tip, chiba needle tip, touhy needle tip, cournand needle tip, mengini needle tip, backcut bevel needle tip, dos santos needle tip, seldinger needle tip, conical tip, curved tip, cutting tip, reverse cutting tip, tapered, tapered cutting tip, micro-point tip, spatula tip, etc.

In some examples, the central lumen extends through the interior of the needle from the proximal end to the tissue penetrating end. For example, the central lumen may extend from an opening at the proximal end and extend along a central axis to an opening on the bevel surface of the tissue penetrating end. In some examples, the central lumen may extend from the proximal end to an opening in the needle at one or more positions locatable proximally to the tissue penetrating end. For example, the central lumen may extend from an opening in the proximal end to an opening at a location along the needle proximal to the tissue penetrating end. In some examples, one or more flat surfaces positioned longitudinally on a length of the needle exterior may be coplanar with an axis of the central lumen.

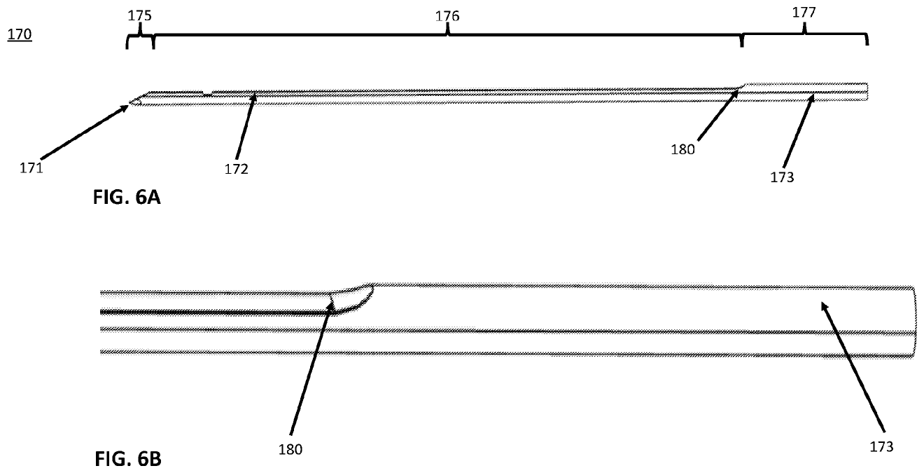

FIGS. 6A and 6B show a side elevation view example of a hypodermic needle 170 as described herein. The distal end 175 has the tissue penetrating tip 171 at a distal end of the needle 170. The tissue penetrating tip 110 is configured to penetrate tissue during use. For example, the tissue penetrating tip 110 may be configured to penetrate multiple layers of dermal tissue and into the vasculature of a patient. A flat surface 172 extends along a cannula of the needle 170 in a flat surface segment 176 segment between the distal end 175 and an engagement segment 177 having an engagement element 173. A detailed expanded view of the engagement segment 177 near the proximal end of the needle 170 is provided to show an example of the transition between the flat surface 172 and the engagement element 173 at the area indicated 180.

This transition segment, region, feature, etc. may be any geometry (e.g., angled, tapered, curved, etc.). In some examples, the transition from a round surface of a needle to a flat surface as shown for example in FIG. 6A or a channel as shown for example in FIG. 1 may include a transition segment that is curved or sloped to the flat surface or channel. In some examples, where the transition segment is angular, the transition segment may provide a ramp or slope at an obtuse angle including the transition segment and the flat surface or channel. In some examples, where the transition segment is angular, the transition segment may provide a ramp or slope at an acute angle including the transition segment and the flat surface or channel. In some examples, where the transition segment is angular, the transition segment may provide a ramp or slope at a right angle including the transition segment and the flat surface or channel.

In some examples, the transition between the engagement element 173 and the flat surface 172 may be configured to facilitate the transition of a tool from a proximal end of the needle 170 (e.g., a medical device, intravascular access device) to the distal end of the needle along the flat surface 172. For example, a guide wire or guide element (not shown) may transition from the intravascular access device along the flat surface 172 and the transition 180 may facilitate the routing of the guide element along the needle.

The engagement element may be any shape configured to engage a tool at a proximal end of the needle. In some examples, the flat surface 172 may extend along the entire length of the needle from the distal end to the proximal end such that the cross-section geometry of the needle is consistent or uniform along the length of the flat surface. In some examples, the needle may be tapered, or otherwise have a cross-sectional geometry that is not uniform along a length of the needle. For example, the needle may taper from a larger proximal end to the distal end such that the needle may dilate an opening in the tissue as it is advanced therethrough.

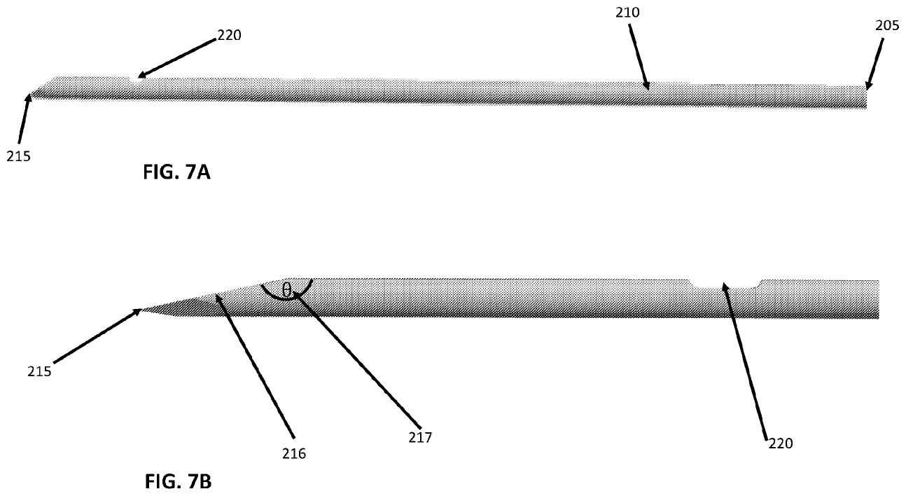

FIGS. 7A and 7B illustrate an example of a hypodermic needle with a detailed expanded view near the distal end. Here, the flat surface 210 can be seen extending from the proximal end of the needle 205 to the distal end of the needle. A notch 220 is shown that intersects the flat surface of the needle. The notch 220 may be positioned anywhere along the length of the needle. In some examples, the needle may have multiple channels orientated in various positions and locations. The notch 220 may be configured to facilitate flow of fluids (e.g., blood, medication, saline, etc.) when the needle is within the patient. The notch 220 may extend into the cannula of the needle to sufficiently expose the central lumen (not shown) extending from the proximal end to the distal end. In some examples, the notch 220 may provide the distal opening of the lumen such that the lumen extends from the distal end to the channel.

In some examples, a hypodermic needle may have a beveled surface 216 extending from the flat surface 210 to the tissue penetrating tip 215. FIG. 7B illustrates an example of an angle 217 between the beveled surface 216 and the flat surface 210. In some examples, the angle 217 may be based on the use and optimized distal end geometry to increase the efficacy of tissue penetration and reduce the physical insult to the tissue as the needle advanced therethrough. In some examples, an increase in angle 217 may provide for an increase in the size of the distal opening of the needle lumen. In some examples, an increase in angle 217 may provide for a decrease in the size of the distal opening of the needle lumen. In some examples, a decrease in angle 217 may provide for an increase in the size of the distal opening of the needle lumen. In some examples, a decrease in angle 217 may provide for a decrease in the size of the distal opening of the needle lumen.

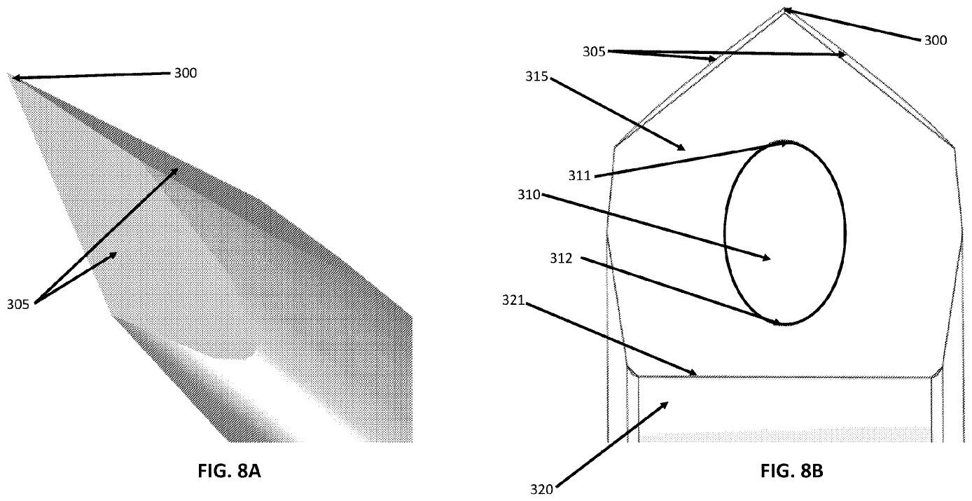

In FIG. 8A and FIG. 8B, detailed views of the needle distal end are provided to shown examples of the tissue penetrating tip 300. The tissue penetrating tip 300 may have an apex that can be the convergence of a plurality of surfaces 305 to optimize the surface area at the distal most point of the tissue penetrating tip 300. The arrangement of the surfaces may be determined during manufacturing based on the optimal arrangement for forming the most effective tip for penetrating tissue. The needle lumen 310 is shown here opening through the distal end generally through a beveled surface 315. The beveled surface 315 is shown here angled towards the tissue penetrating tip 300 from the flat surface 320. The departure of the beveled surface 315 at point 321 may be at any angle to optimize the function of the tissue penetrating tip 300 to penetrate through tissue.

In some examples, the flat surface may be positioned along a length of the needle generally opposite of the tissue penetrating tip. The examples in FIGS. 8A and 8B illustrate this, where a midline may be drawn from the tissue penetrating tip 300 and a midline of the flat surface 320. In this example, either side of the midline may be considered generally symmetrical to the other. In some examples, the flat surface 320 may be positioned such that a midline of the needle provides asymmetrical sides. For example, the flat surface may be positioned longitudinally along the needle at any position around the needle perimeter. For example, the central axis of the needle may have determinable radials extending outward therefrom. The flat surface may be positioned perpendicular to any radial extending outwards from the needle's central axis. In some examples, the flat surface may be more proximal to the tissue penetrating tip than at an opposite end of a diameter of the central lumen.

FIG. 8B also provides a detailed view of exemplary geometry of the distal opening of the needle lumen 310. Here, the example shows a generally oval shape having a distal rim 311 and a proximal rim (e.g., needle heel) 312. The length (e.g., long axis) extending between the distal rim 311 and heel 312 may be based on the angle of the beveled surface 315. A short axis, generally perpendicular to the long axis may have a length related to the diameter of the needle lumen 310. In some examples, the distal opening of the needle lumen may have a predetermined geometry such that the beveled surface 315 may be created at an angle relative to the flat surface 320 to increase or decrease the long axis length. The heel 312 may be configured to reduce or prevent coring.

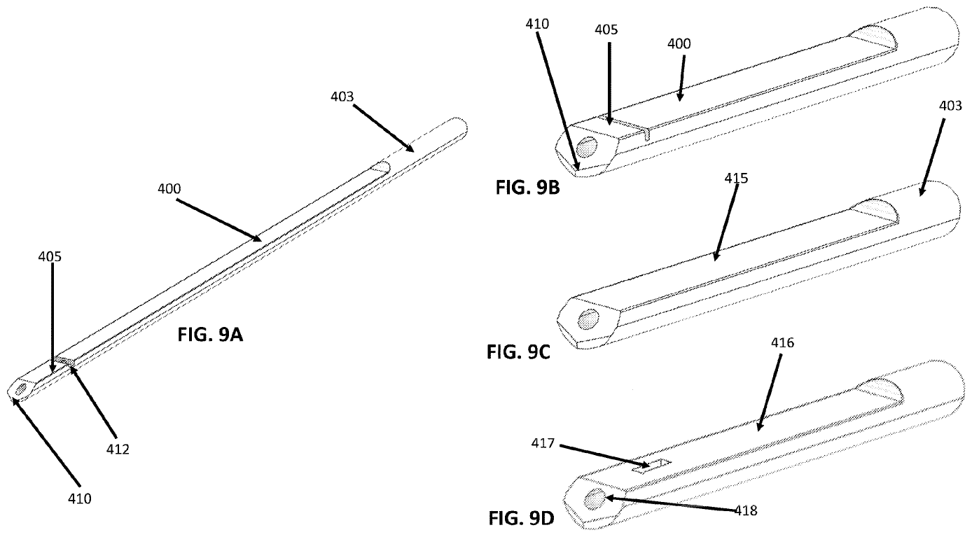

In some examples, a hypodermic needle may have more than one flat surface. For example, a needle may have more than one flat surface longitudinally positioned along the needle. In some examples, the flat surface may be separated by a channel such that the flat surface has multiple segments along the same plane. FIG. 9A illustrates a needle having a first flat surface segment 400 and a second flat surface segment 405. The first flat segment 400 is shown extending from the engagement element 403 to the notch 412 and a second flat surface segment 405 then extends from the distal side of the notch 412 to the distal end of the needle. FIG. 9B also illustrates an example of how a needle flat surface may be separated by a notch. In this example, each flat surface segment may be coplanar with one another. FIG. 9C illustrates another example of a needle having a continuous flat surface 415 extending from the engagement element 403 to the distal end. Yet another example of a notch configuration for needles described herein is illustrated in FIG. 9D where the notch 417 is positioned on the flat surface 416. Here, the notch 417 is shown to align with the lumen 418 and configured to indicate bleed-back when the tissue penetrating tip has been inserted into a vessel.

The arrangement of the flat surfaces and flat surface segments may be based on the use of the needle and/or the combination of the needle with a tool or other medical device such as an intravascular access device. For example, the arrangement of the flat surface position on the needle may be based on the routing of a guide element or guide wire extending from a medical device at or near the proximal end of the needle to the distal end of the needle.

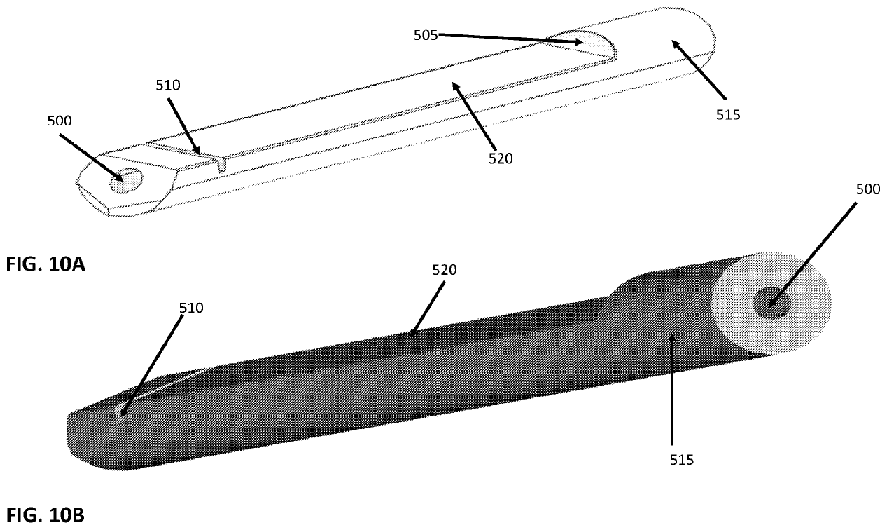

FIG. 10A and FIG. 10B provide perspective views of a hypodermic needle showing the central lumen 500 extending from the proximal end of the needle to the distal end through the beveled surface proximal to the tissue penetrating tip. A channel 510 is positioned near the distal end of the needle and may expose the central lumen 500 and be configured to allow fluid flow outside of the central lumen 500. In some examples, the channel may be configured to provide a visible flashback of blood when the tissue penetrating tip is inserted into a blood vessel. For example, when the needle is used with a catheter, such as a catheter coupled to an intravascular access device, the needle may be sufficiently within the central lumen of the catheter. When the needle is advanced into a blood vessel, blood may flow from the opening of the needle lumen 500 proximally within the needle. Visible confirmation of vascular insertion may be required for the user to appreciate the placement of the needle during use. Accordingly, the blood may enter the opening of the needle lumen (e.g., on the bevel surface of the needle distal end) and flow out of the lumen exposed by the channel 510. The blood flowing out of the lumen exposed by the channel 510 may be retained within the catheter central lumen but visible therein by the user.

FIG. 10A also provides a view of exemplary geometry for the transition from the needle proximal end 515 and the flat surface 520. In some examples, this transition 505 may be an angular geometry with a slope from the proximal end 515 to the flat surface 520. Examples of the slope transition at area 505 may be flat, substantially flat, curved. In some examples, the geometry may be a vertical or substantially vertical transition. In some examples, the transition 505 may be a tapered transition from the proximal end to the flat surface. The transition 505 may have any geometry for the transition from the proximal end to the flat surface.

The type of transition may be based on the engagement of the proximal end of the needle with a tool or device such as a needle carrier for an intravascular access device. In some examples, the transition 505 may be configured to facilitate the transition of a guide element (e.g., guide wire) that can be advanced along the needle exterior including from the proximal end of the needle and along the flat surface to beyond the distal tip of the needle. In some examples, the guide element may be configured to route along the needle exterior including routing along the transition from the proximal end to the flat surface.

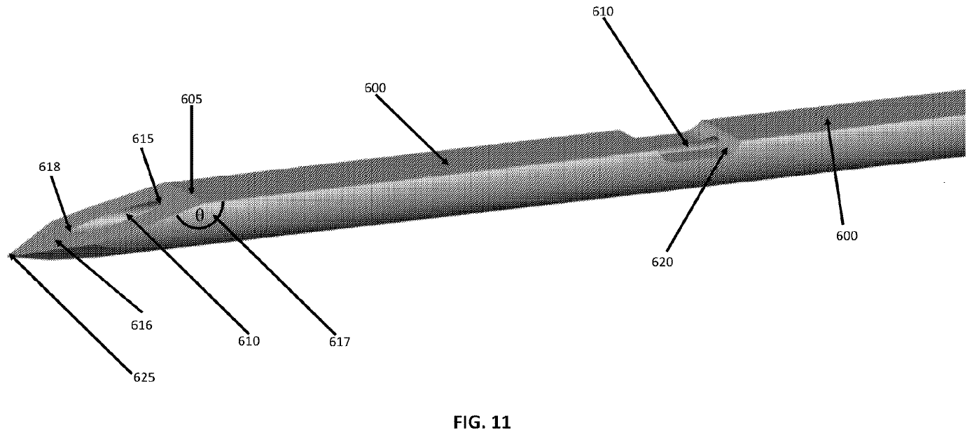

FIG. 11 shows an exemplary perspective view zoomed in on the distal end of a hypodermic needle as described herein. The flat surface 600 can be seen extending along a length of the distal end of the needle and may be a discontinuous surface where the channel 620 is cut into the needle. In some examples, a discontinuous flat surface may be coplanar with each section or segment of flat surface along the needle. In some examples, a discontinuous flat surface may not be coplanar with each section or segment of flat surface along the needle. In some examples, each section or segment of flat surface may be coplanar, not coplanar, or a combination thereof relative to one or more other flat surface sections of segments along the needle. The channel 620 is cut to expose the needle lumen 610 such that fluid (e.g., blood) may flow from within the needle lumen 610 and be visible at an area defined by the channel 620. The exposure of blood by the channel 620, when the needle is in use may be configured to indicate appropriate placement of the needle within a blood vessel. For example, flashback or a flash of blood exposed by the channel may indicate the tissue penetrating tip 625 and needle lumen distal opening 610 are sufficiently within the blood vessel.

The beveled surface 616 extends from the flat surface 600 at a transition area 605 and at an angle 617. The angle 617 may be configured to determine the size and geometry of the distal opening of the needle lumen 610 on the beveled surface 616. Here, the long axis length between the distal rim 618 and the heel 615 may be relative to the angle 617 of the beveled surface 616. The geometry of the needle lumen distal opening may be configured to facilitate a flow rate of blood or other fluid passing therethrough. For example, a fluid passing through the needle lumen 620 may have one or more attributes (e.g., viscosity, density, heterogeneity, etc.) that can flow through the needle lumen at a more advantageous rate based on the distal opening of the needle lumen 610.

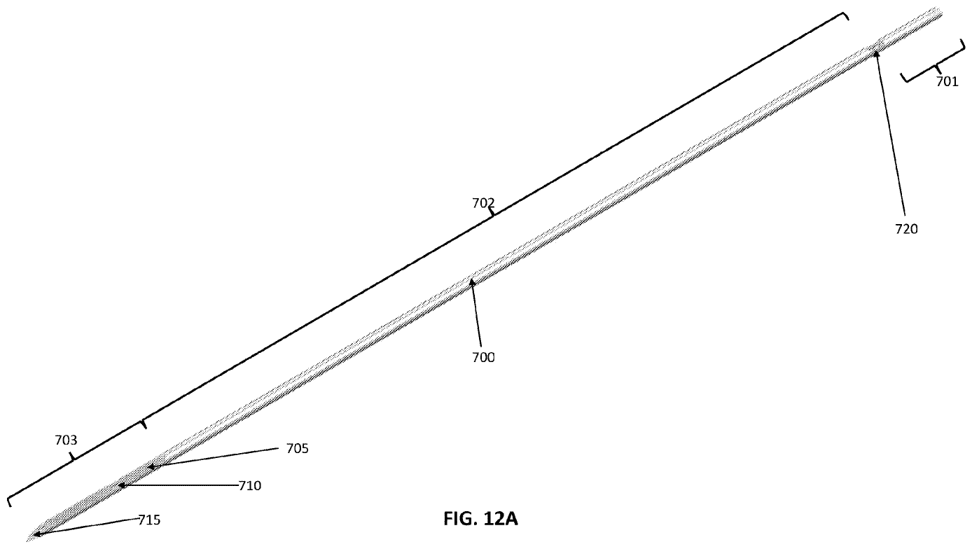

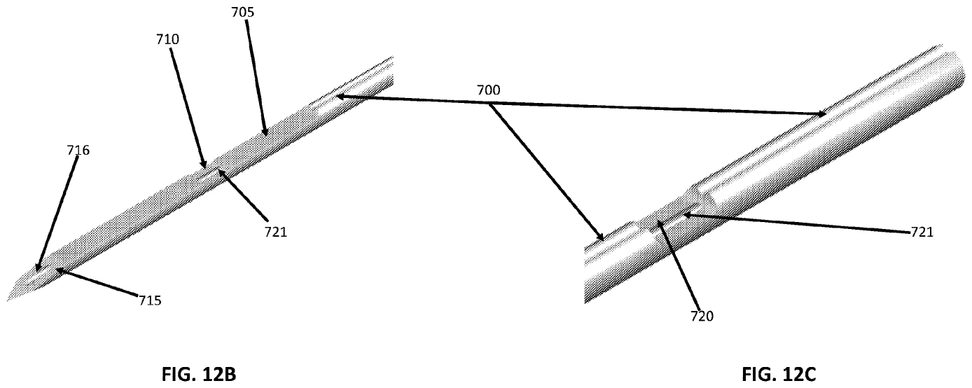

FIGS. 12A to 12C illustrate another example of a hypodermic needle as described herein. In this example, the needle may be discussed relative to one or more segments. For example, the proximal segment 701 may have a length extending distally from the proximal end of the needle to one or more channels 720. A midsegment 702 may comprise a length extending distally from the notch 720 to the distal segment 703 that can comprise the flat surface 705 and the distal tip with the beveled surface 715. A second notch 710 is shown locatable on the flat surface near the distal end of the needle. Here, along a routing element extends from the proximal end 701 to the proximal end of the flat surface 705. As illustrated here, the routing element 700 is a relief into the needle that can be configured to route, receive, retain, guide, etc. one or more elongate members positioned therein. For example, a guide wire (not shown) may have a length of elongate body that can be configured to seat or otherwise engage the routing element 700. The elongate body of the guide wire may be coupled to a guide element at a distal end configured to contact the flat surface 705 of a hypodermic needle described herein. In some examples, the flat surface of a hypodermic needle described herein may be configured with dimensions corresponding to the dimensions of the guide element at the distal end of the guide wire. For example, the guide element may have a geometry complementary to the distal end of the needle such that the guide element has a corresponding flat surface configured to operably contact the flat surface of a hypodermic needle described herein. In some examples, the guide element may seat on the flat surface 705 in a ready-to-use configuration as the needle is inserted into a blood vessel. The guide element may be advanced distally along the flat surface 705 as the guide wire is advanced from the proximal end. In some examples, the transition geometry from the needle to the flat surface may correspond to a geometry of the proximal end of the guide element to promote engagement of the guide element when seated on the needle flat surface 705.

In some examples, the guide element may be positioned on the flat surface (e.g., 705) of a hypodermic needle described herein. The notch (e.g., 710) may be sufficiently cut into the needle to expose the needle lumen such that blood may flow from the needle lumen near the channel and between the channel and surface of the guide element. Any of the notches described herein may be positioned on any surface of the needle and at any position along a length of the needle. For example, a notch may be positioned adjacent to the distal end of the needle. A notch may be positioned between the distal end of the needle and the proximal end of the needle. For example, a notch may be positioned 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, or greater. Needles having multiple notches may include notches positioned at different distances relative to the distal end of the needle (e.g., tissue penetrating tip) and may be positioned on any surface of the needle relative to the flat surface, channel, or round surface of the needle (e.g., elongate body).

In some examples, a guide element in operable communication with a hypodermic needle described herein may have complementary geometry to the needle such that when the guide element is positioned on the flat surface of the needle, the combination of the guide element and the needle distal end (e.g., including the flat surface) may form a generally cylindrical combined cross-sectional geometry. In some examples, the guide element and the needle are configured to be operably coupled with one another and configured to be locatable within a catheter lumen. For example, a hypodermic needle described herein, and the guide element may be positioned within a catheter at a distal end of an intravascular access device.

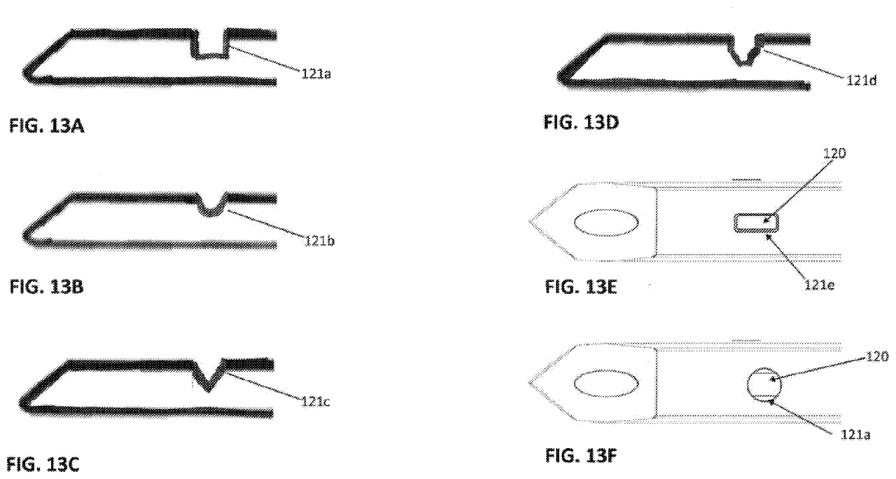

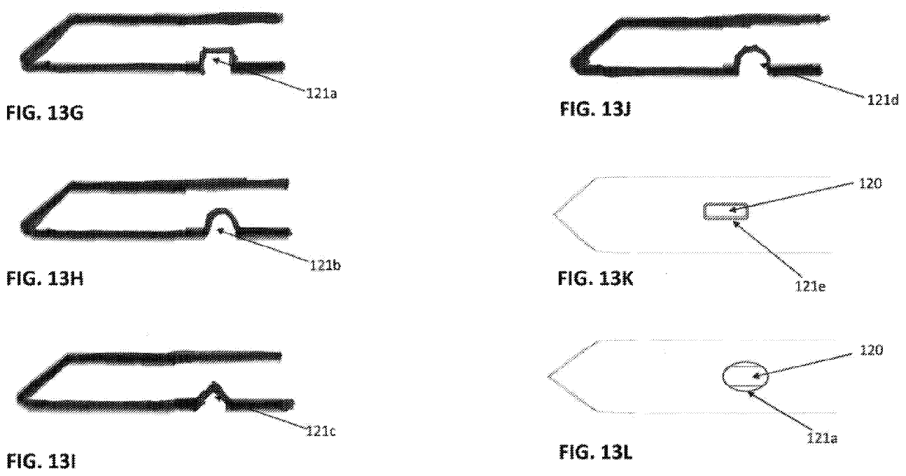

Referring to FIG. 12B and FIG. 12C the distal end in FIG. 112B is detailed in to show the notch 710 exposing the needle lumen 721. The beveled surface 715 can be seen with the distal opening of the needle lumen 716 having a long axis with a length that may be defined by the angle of the beveled surface 715 and the flat surface 705. The routing element 700 can be seen on both FIG. 13A and FIG. 13B as a longitudinal concave depression into the needle. Considering FIG. 13A, the routing element 700 extends generally colinear with the needle lumen and directly through the transition to the flat surface 705. In this way, a guide wire may extend through the routing element 700 and into a guide element coupled to the distal end of the guide wire. The guide element may then be operably positioned on the flat surface 705 such that the proximal end of the guide element may be in contact with the needle along the flat surface 705 including the proximal transition from the flat surface 705.

The notch 720 detailed in FIG. 13B is shown to expose the needle lumen 721. In some examples, a hypodermic needle described herein may have one or more notch along a length of the needle. In some examples, the channel may be located along any arc, side, position, etc. of the needle. One or more of the notches may be located perpendicular to the plane of the flat surface as illustrated in FIG. 13A. In some examples, one or more notches may be positioned or cut at an angle relative to the plane defined by the flat surface of the needle. The position of one or more notches may be configured to provide the most advantageous visibility for blood to be seen flowing from the needle lumen at the channel. In some examples, the notch may be on an opposite side of the needle relative to the flat surface such that the needle may be inserted with the flat surface facing away from the user while the notch positioned on the opposite side of the needle may still be visible to the user.

FIGS. 13A to 13L illustrate several examples and variations of notch configurations for needles described herein. It is to be appreciated that in some embodiments where the needle is adapted and configured for use in a vascular access device, the notch may be shaped, sized and positioned relative to the distal tip to aid in detection of blood flow-so called flashback or blood detection or indication that the distal tip and lumen are in a blood vessel. In some embodiments, the notch is sized and shaped and spaced relative to the distal end to perform a flashback function.

Referring to FIGS. 13A to 13D, detailed views of hypodermic needle distal sections are shown in a cross-section view to illustrate examples of notch geometry creating an opening through the elongate body (e.g., needle wall) to expose the lumen extending through the needle. For example, FIG. 13A, the notch 121a is generally square and positioned perpendicular across the elongate body with a sufficient depth cut away from the needle wall to expose the needle lumen. Similarly, notches 121b, 121c, 121d all may be perpendicular to the needle body. In FIG. 13E a top plan view of a needle distal section shows the notch 121e generally parallel to the needle lumen and positioned on the needle flat surface or channel to expose the needle lumen therethrough. FIG. 13F also shows a notch 121f positioned on the flat surface or channel of the needle with a circular geometry from the top plan view.

Additional examples of notch configuration and position are shown in FIGS. 13G to 13L where the notches here are now positioned on a bottom surface of the needle.

Referring to FIGS. 13G to 13J, notches extending perpendicular to the needle body are shown with various geometric cross sections on a bottom surface of the needle (e.g., the surface adjacent the tissue penetrating tip). In FIGS. 13K and 13L, the bottom-plan view of these examples shows notch configurations exposing the lumen without extending perpendicular to the needle body.

In any example of a needle described herein, the flat surface may be a flat surface on a length of the needle. In some examples, the flat surface extends from the distal end of the needle to the proximal end of the needle. In some examples, the flat surface may extend along a length or segment of the needle from the proximal end of the needle. In some examples, the flat surface may extend along a length or segment from the distal end of the needle. In some examples, the flat surface may extend along a length of the needle between the proximal end of the needle and the distal end of the needle. In some examples, the distal end of the needle. In some examples, the distal end of the needle may include the beveled surface. In some examples, the distal end of the needle may be the distal end of the flat surface.

In any example of a needle described herein, the flat surface may be a concave surface on a length of the needle. The concave surface may be concave between a width of the concave surface extending laterally relative to the needle. In some examples, the concave surface may be concave into the needle material. In some examples, cross sectional geometry of the needle may comprise the needle being generally annular around the needle lumen with the concave surface segment of the needle being thinner than the rest of the needle. In some examples, the concave surface extends from the distal end of the needle to the proximal end of the needle. In some examples, the concave surface may extend along a length or segment of the needle from the proximal end of the needle. In some examples, the concave surface may extend along a length or segment from the distal end of the needle. In some examples, the concave surface may extend along a length of the needle between the proximal end of the needle and the distal end of the needle. In some examples, the distal end of the needle. In some examples, the distal end of the needle may include the beveled surface. In some examples, the distal end of the needle may be the distal end of the concave surface.

In any example of a needle described herein, the flat surface may be a convex surface on a length of the needle. The convex surface may be concave between a width of the convex surface extending laterally relative to the needle. In some examples, the convex surface may be convex outward from the needle material. In some examples, cross sectional geometry of the needle may comprise the needle being generally annular around the needle lumen with the convex surface segment of the needle being thicker than the rest of the needle. In some examples, the convex surface extends from the distal end of the needle to the proximal end of the needle. In some examples, the convex surface may extend along a length or segment of the needle from the proximal end of the needle. In some examples, the convex surface may extend along a length or segment from the distal end of the needle. In some examples, the convex surface may extend along a length of the needle between the proximal end of the needle and the distal end of the needle. In some examples, the distal end of the needle. In some examples, the distal end of the needle may include the beveled surface. In some examples, the distal end of the needle may be the distal end of the convex surface. In some examples, the needle lumen may have a larger diameter along a length of the needle corresponding to the length of the convex surface. In some examples, the larger diameter of the needle lumen may be configured to facilitate an increased flow (e.g., flow rate) of fluid flowing therethrough.

In some examples, a needle described herein may have a channel that extends across (e.g., laterally) the needle at any position along the needle length. As described herein, a needle may comprise one or more channels at any position of the needle. Although images provided herein illustrate examples of a channel as an elongate section of the needle removed (e.g., cut away), a channel may include any opening, aperture, depression, etc. having any geometric configuration. For example, a channel may have one or more circular openings (e.g., holes) extending through the needle from the exterior surface into the needle lumen.

A method of manufacturing a needle described herein may include determining a location of a flat surface on the cannula (e.g., needle body). The location of the flat surface may include establishing a longitudinal placement, dimensions, and characteristics of the flat surface. The needle may initially have a generally cylindrical exterior surface prior to removing material at the determined flat surface location. The needle may be positioned in a cassette configured to retain the needle during the removal process. One or more methods of removing material from the needle body at the determined location of the flat surface may then be applied to the needle. The step of removing material may continue until the flat surface is established along the determined location.

In some examples, the cassette may be configured to retain a plurality of needles. For example, more than one needle may be processed at a time by positioning (e.g., inserting) the needles in or on the cassette such that the removing method may effectively remove or modify the cannula to provide the flat surface thereon.

In some examples, methods of removing material from the needle surface to provide the flat surface may include milling, grinding, laser processing (cutting, welding, marking), burr free cutting, laser welding, micro welding, micromachining, injection molding, coating, swaging, brazing, forming, bending, electropolishing, electro-chemical marking, electro-discharge machining (EDM), swagging, flattening, grinding, etc. In some examples, any milling, machining, physical, electrical, chemical process configured to provide a flat surface as described herein may be used.

In some examples, cutting the exterior surface of the needle may include removing an arc segment of the needle exterior surface. In some examples, the needle may have one or more segments that can be modified, cut, adjusted, or otherwise changed based on a predetermined configuration of the needle. For example, an engagement segment at a proximal end may be generally cylindrical, or of any geometry configured for use with an additional tool or element operably engageable thereto. In some examples, manufacturing a flat surface as described herein may include extrusion techniques where a needle is passed through one or more dies having a shape being configured to modify the exterior of the needle cannula. For example, a needle being generally cylindrical may be inserted into an extrusion apparatus and passed through one or more die configured to change the cylindrical exterior of the needle to a needle having a flat surface as described herein. In some examples, providing a flat surface as described herein may be achieved using a press or mold where a needle with a starting configuration having a generally cylindrical cannula along the length of the needle is inserted or operably positioned in or on a press. A mold or die may be applied based on the location and configuration desired for the resulting flat surface or needle surface configuration. Pressure may be applied to modify the shape of the needle according to the determined location of the flat surface as provided by the mold or die.

In some examples, a needle described herein may be configured to reduce the pain or impact on a patient during use. For example, the tissue penetrating tip, flat surface or other element of a needle described herein may reduce the pain felt by a patient. In some examples, a needle described herein may reduce the amount of pain as determined by a Visual Analog Scale, A visual analog scale may be a linear scale used to assess pain by a range of numbers (e.g., 0 -10 or 0-100). For example, the smaller numbers can be indicative of low or absence of pain and the higher numbers can be indicative of increased pain. In some examples a needle described herein may be graded on a visual analog scale a lower number compared to another needle. In some examples, pain may be determined by the Gracely Box Scale using words corresponding to numbers. The Gracely Box Scale can be used to detect differences in different regions of a pain scale, Some examples of words used for this scale may be painless, weak, mild pain, moderate pain, etc. In some examples, a needle described herein may be described with words on the Gracely Box Scale indicating less pain than one or more other needles.

In some examples, a needle described herein may be configured to reduce the amount of force required to penetrate or perforate tissue. For example, a needle described herein may have a tissue penetrating tip configured to reduce the amount of force required to penetrate the skin by 1%, 5%, 10%, 15%, 20%, 25%, 30%, 35%, 40%, 45%, 50%, 55%, 60%, 65%, 70%, 75%, 80%, 85%, 90%, 95%, 100%, or any percentage therebetween.

Additional aspects of the construction and operation of a catheter placement device which includes a housing or handle having mechanism for advancing a guide structure or guide element which carries the catheter where the handle is adapted to automatically retract both the access needle and the guide structure or guide element from the catheter after the placement procedure is complete, a button activated automatic needle and guide withdrawal assembly are described in U.S. Patent Publication No. 2008/0300574 and U.S. U.S. Pat. No. 9,522,254, each of which is incorporated herein by reference in their entirety.

Still other details of representative intravascular catheter insertion devices and methods are described in U.S. Pat. Nos. 5,704,914 and 5,800,395 and in U.S. Patent Publication Nos. 2010/0094310; U.S. 2010/0210934; and U.S. 2012/0197200, the full disclosure of each of these are incorporated herein by reference in their entirety.

There may be one or more variations, alternatives, constructions, compositions, and/or components described herein that can be used to modify an element, component, device, system, process, etc. of a guide element, intravascular access device and/or an associated structure or process. Accordingly, any variation, description, example, element, component, process, method, method step, etc. described herein can be used as a modification, variation, and/or alternative to any element, device, system, composition, example, component, process, method, method step, etc. described in provisional patent application No. 63/328,732 filed on April 7, 2022, entitled “INTRAVASCULAR CATHETER WITH INTEGRATED GUIDE STRUCTURE”; and/or PCT application number PCT/US2021/54046 filed on Oct. 7, 2021, entitled “INTRAVASCULAR CATHETER WITH INTEGRATED GUIDE STRUCTURE” the entireties of which are incorporated herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A hypodermic needle comprising:
an elongate body having a proximal end and a distal end;
a tissue penetrating tip at the distal end;
a lumen extending through the elongate body from the proximal end to the distal end; and
a channel formed along a length of the elongate body.

2. The hypodermic needle of claim 1, wherein the channel is formed by a press fit depression in a surface of the elongate body.

3. The hypodermic needle of claim 2, wherein the lumen adjacent to the channel has a complementary shape to the depression in a surface of the elongate body.

4. The hypodermic needle of claim 1, further comprising a transition segment positioned between a proximal length of the elongate body and the channel.

5. The hypodermic needle of claim 1, further comprising a transition segment positioned at a proximal end of the elongate body, wherein the channel extending proximally from the transition segment is concave relative to the length of elongate body proximal to the transition segment.

6. The hypodermic needle of claim 1, further comprising a proximal transition segment, a distal transition segment, wherein the channel further comprises a proximal channel and a distal channel further wherein the distal transition segment is positioned between the proximal channel and the distal channel.

7. The hypodermic needle of claim 1, wherein the elongate body further comprises a proximal region and a distal region wherein the proximal region and the distal region are separated by a transition segment.

8. The hypodermic needle of claim 4, wherein the transition segment comprises a surface forming an acute angle with a surface of the channel.

9. The hypodermic needle of claim 1, wherein the channel extends along an entire length of the elongate body.

10. The hypodermic needle of claim 7, wherein a transition segment separates the proximal channel from the distal channel, wherein the distal channel is more concave than the proximal channel.

11. The hypodermic needle of claim 1, wherein the lumen is non-circular.

12. The hypodermic needle of claim 1, further comprising a notch extending through the elongate body into a portion of the lumen.

13. The hypodermic needle of claim 1, wherein the tissue penetrating tip is positioned on the distal end of the elongate body opposite the channel.

14. The hypodermic needle of claim 1, the channel comprising a distal channel and a proximal channel, wherein the distal channel extends between the elongate body distal end and a transition segment, and the proximal channel extends from the transition segment to the elongate body proximal end wherein the depth of the distal channel is greater than the depth of the proximal channel.

15. The hypodermic needle of claim 1, wherein the channel is crescent shaped and formed in an outer wall of the elongate body and the lumen has a corresponding crescent shape to the channel crescent shape.

16. A hypodermic needle comprising:
an elongate body having a proximal end and a distal end;
a tissue penetrating tip positioned at the distal end;
a lumen extending from the proximal end to the distal end;
a proximal channel extending along a proximal portion of the elongate body; and
a distal channel extending from a distal portion of the elongate body to a transition
segment that separates the proximal channel from the distal channel.

17. The hypodermic needle of claim 17, wherein relative to an outer wall of the elongate body the distal channel to is deeper than the proximal channel.

18. The hypodermic needle of claim 16, further comprising a proximal transition segment at the proximal end of the proximal channel separating the proximal channel from an outer wall of the elongate body.

19. The hypodermic needle of claim 16, wherein the proximal channel and the distal channel are pressed into the elongate body.

20. The hypodermic needle of claim 16, further comprising a proximal transition segment, a distal transition segment and an annular shaped proximal portion of the elongate body adjacent to the proximal transition segment.

21.-37. (canceled)
